(19) United States
(12) Patent Application Publication
(10) Pub. No.: US 2008/0159969 A1
(43) Pub. Date: Jul. 3, 2008

(54) Pharmaceutical Or Cosmetic Composition Comprising Mequinol And At Least One Depigmenting Agent

(75) Inventors: Isabelle Pelisson, Vallauris (FR); Andre Jomard, Saint Vallier de Thiey (FR); Cecile Cousin, Pegomas (FR)

Correspondence Address: OBLON, SPIVAK, MCCLELLAND MAIER & NEUSTADT, P.C., 1940 DUKE STREET, ALEXANDRIA, VA, 22314, US

(73) Assignee: Galderma Research & Development, S.N.C., Valbonne Sophia Antipolis (FR)

(21) Appl. No.: 11/596,356

(22) PCT Filed: May 13, 2005
(86) PCT No.: PCT/FR05/01211

§ 371 Date: Aug. 20, 2007

(30) Foreign Application Priority Data
May 14, 2004 (FR) 0405280

(51) Int. Cl. A61K 8/30; A61K 31/351; A61K 31/20; A61Q 17/04; A61P 17/00; A61Q 19/02; A61K 31/19; A61K 31/375

(52) U.S. Cl. 424/59; 424/62

(57) ABSTRACT

The invention relates to a pharmaceutical or cosmetic composition comprising, in a physiologically acceptable medium, mequinol and at least one depigmenting agent, and to the use thereof.

The present invention relates to a novel cosmetic or dermatological composition comprising a combination of mequinol (or 4-hydroxyanisole) and a depigmenting agent that can be used to depigment the skin via topical application. The composition according to the invention may also contain a sunscreen.

Pigmentation of the skin results from the synthesis of melanin in the melanocytes of the dermis by the transformation of tyrosine under the influence of a cupro-protein enzyme, tyrosinase.

A skin depigmenting effect may thus be obtained by using a compound that degrades melanin or a tyrosinase inhibitor the blocks the mechanism of transformation of tyrosine into melanin.

Mequinol, or 4-hydroxyanisole, has been known for a long time as a compound with depigmenting activity (Brun R., Journal of the Society of Cosmetic Chemists (1960), 11, 571-9).

The mode of action of mequinol as a depigmenting agent is to act as a tyrosinase substrate (Riley P., Journal of Pathology (1969), 97(2) 1185-91 and Journal of Pathology (1969), 97(2), 193-206).

Vitamin C or derivatives thereof are usually used as antioxidants at concentrations of less than 0.005% of the entirety of the composition, as illustrated in patent U.S. Pat. No. 6,353,029.

Kojic acid is already known in the prior art as a fungal metabolic agent with antioxidant properties, which causes contact dermatitis when its use is prolonged.

Azelaic acid has been reported in the literature as being effective in the treatment of melasma, but this activity appears to be controversial (Sarkar et al., Dermatology 2002, 205: 249-254).

The Applicant has discovered, surprisingly, that the combination of mequinol and of a depigmenting agent taken from a precise list makes it possible to obtain, by means of a synergistic effect, a depigmenting response that is much more efficient than that obtained with mequinol alone, with substantially lower doses.

These smaller amounts will also make it possible to significantly reduce the harmful side effects (redness and irritation) associated with the use of a depigmenting agent.

The invention therefore relates to a skin depigmenting composition comprising, in a physiologically acceptable medium, mequinol and at least one depigmenting agent chosen from the list consisting of kojic acid and derivatives thereof; azelaic acid and derivatives thereof; dicarboxylic acids; vitamin C and derivatives thereof, arbutin and derivatives thereof, and paracetamol.

In particular, the skin depigmenting composition may comprise, as depigmenting agent, kojic acid, azelaic acid or arbutin.

The term “physiologically acceptable medium” means a medium that is compatible with the skin, mucous membranes and/or the integuments.

The term “depigmenting agent” means any active agent with depigmenting activity on the skin. This activity makes it possible to reduce the already-existing pigmentation of the skin and also to prevent any additional pigmentation greater than the natural pigmentation.

The application of a composition containing the combination according to the invention makes possible to obtain pronounced bleaching of the skin.

The invention also relates to a skin depigmenting composition comprising, in a physiologically acceptable medium, mequinol, at least one depigmenting agent and at least one sunscreen.

The composition according to the invention advantageously comprises between 0.0001% and 20% by weight of mequinol relative to the total weight of the composition and between 0.025% and 20% by weight of depigmenting agent relative to the total weight of the composition, and preferably, respectively, between 0.001% and 10% by weight of mequinol relative to the total weight of the composition and between 0.05% and 10% by weight of depigmenting agent relative to the total weight of the composition.

The composition may also comprise at least one sunscreen in preferential concentrations ranging from 0.001% to 30.00% by weight relative to the total weight of the composition.

Among the sunscreens that may be mentioned as non-limiting examples are physical sunscreens such as titanium dioxide and zinc oxide, and chemical sunscreens such as octocrylene, ethylhexyl methoxy-cinnamate, octyl salicylate, avobenzone, oxybenzone, ecamsule and drometrizole trisiloxane, or mixtures thereof.

In one embodiment variant of the present invention, the composition may comprise several sunscreens, in which case each sunscreen may be added in a concentration ranging from 0.001% to 20% by weight relative to the total weight of the composition.

The compositions according to the invention may also comprise any additive usually used in cosmetics or pharmaceuticals, such as sequestrants, antioxidants, preserving agents, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents. Needless to say, a person skilled in the art will take care to select this or these optional additional compound(s), and/or the amount thereof, such that the advantageous properties of the composition according to the invention are not, or are not substantially, adversely affected.

These additives may be present in the composition in a proportion of from 0.001% to 20% by weight relative to the total weight of the composition.

A subject of the present invention is also a pharmaceutical composition as described above, as medicament.

A subject of the present invention is also a cosmetic composition.

The compositions containing the combination according to the invention may be in any galenical form normally used for topical application, for example in the form of solutions, gels or dispersions of the lotion or serum type, of emulsions of liquid or semi-liquid consistency of the milk type, obtained by dispersing a fatty phase in an aqueous phase (O/W) or conversely (W/O), or of suspensions or emulsions of soft, semi-solid or solid consistency of the cream or gel type, or alternatively of microemulsions, microcapsules, microparticles or vesicular dispersions of ionic and/or nonionic type. These compositions are prepared according to the usual methods.

In a known manner, the cosmetic or dermatological compositions of the invention may contain adjuvants that are common in cosmetics or dermatology, such as emulsifiers, hydrophilic or lipophilic gelling agents, hydrophilic or lipophilic active agents other than titanium oxide and α-hydroxy acids, preserving agents, antioxidants, fragrances, fillers, screening agents and dyestuffs. The amounts of these various adjuvants are those conventionally used in cosmetics and/or dermatology, for example from 0.01% to 20% of the total weight of the composition. Depending on their nature, these adjuvants may be introduced into the fatty phase, into the aqueous phase and/or into the lipid vesicles.

The invention also relates to the use of the composition according to the invention as described above in pharmaceuticals and cosmetics.

The compositions of the invention are particularly suitable for treating and preventing pigmentation disorders. Preferentially, the compositions of the invention are suitable for treating and preventing hyperpigmentation disorders such as melasma, chloasma, lentigo, senile lentigo, vitiligo, freckles, post-inflammatory hyperpigmentation caused by abrasion, a burn, a scar, a dermatitis or a contact allergy; nevi, hyperpigmentations of genetic origin, hyperpigmentations of metabolic or medicinal origin, melanomas or any other pigmentary lesions, especially those induced by photo-induced or chronological ageing of the skin and of the integuments.

More preferentially, the compositions according to the invention are suitable for treating melasma.

The compositions according to the invention also find an application in cosmetics, in particular in protecting against the harmful aspects of sunlight, in particular against pigmentary marks.

The invention also relates to a non-therapeutic cosmetic treatment process for beautifying the skin and/or for enhancing its surface appearance, characterized in that a composition comprising mequinol and at least one depigmenting agent is applied to the skin and/or its integuments.

The formulation examples below illustrate the compositions according to the invention, without, however, limiting its scope. Examples illustrating the depigmenting activity of the various compositions according to the invention are also described.

In the compositions below (Examples 1 to 6), the proportions of the various constituents are expressed as weight percentages relative to the total weight of the composition.

### EXAMPLE 1

| | |
|---|---|
| Mequinol | 0.1 |
| Azelaic acid | 0.1 |
| Steareth-1 | 3 |
| Steareth-21 | 2 |
| Caprylic/capric triglyceride | 4 |
| Isohexadecane | 5 |
| Cetearyl alcohol | 1 |
| Stearic acid | 1.5 |
| Sodium sulfite | 0.2 |
| Propylene glycol | 8 |
| Glycerol | 2 |
| Phenoxyethanol | 1 |
| Purified water | qs 100 |

### EXAMPLE 2

| | |
|---|---|
| Mequinol | 0.1 |
| Kojic acid | 0.2 |
| Carbomer | 0.1 |
| Methyl paraben | 0.18 |
| Propyl paraben | 0.02 |
| Cetyl alcohol | 1 |
| Stearic acid | 0.8 |
| Caprylic/capric triglyceride | 1.5 |
| Cyclomethicone | 1 |
| Mineral oil | 2 |
| Propylene glycol | 5 |
| Glycerol | 2 |
| Triethanolamine | 5 |
| Purified water | qs 100 |

### EXAMPLE 3

| | |
|---|---|
| Purified water | qs 100 |
| Methyl paraben | 0.20 |
| Glycerol | 5 |
| Glyceryl stearate/PEG-100 | 2.30 |
| stearate | |
| Glyceryl stearate/sodium | 8.00 |
| lauryl sulfate | |
| Cetearyl alcohol/Ceteareth-20 | 1.00 |
| Mineral oil | 9.00 |
| Propyl paraben | 0.10 |
| Sodium metabisulfite | 0.15 |
| Mequinol | 0.1 |
| Arbutin | 2 |
| Triethanolamine | qs pH 4.5 |

### EXAMPLE 4

| | Amounts in % |
|---|---|
| Starting materials | weight for weight |
| Purified water | qs 100 |
| Methyl paraben | 0.20 |
| Glycerol | 5 |
| Glyceryl stearate/PEG-100 stearate | 2.30 |
| Glyceryl stearate/sodium lauryl | 8.00 |
| sulfate | |
| Cetearyl alcohol/Ceteareth-20 | 1.00 |
| Mineral oil | 9.00 |
| Avobenzone | 2 |
| Zinc oxide | 2 |
| Propyl paraben | 0.10 |
| Sodium metabisulfite | 0.15 |
| Mequinol | 2 |
| Vitamin C | 0.06 |
| Triethanolamine | qs pH 4.5 |

### EXAMPLE 5

| | Amounts in % |
|---|---|
| Starting materials | weight for weight |
| Purified water | qs 100 |
| Methyl paraben | 0.20 |
| Glycerol | 5 |
| Glyceryl stearate/PEG-100 stearate | 2.30 |
| Glyceryl stearate/sodium lauryl | 8.00 |
| sulfate | |
| Cetearyl alcohol/Ceteareth-20 | 1.00 |
| Mineral oil | 9.00 |
| Avobenzone | 2 |
| Octocrylene | 10 |
| Propyl paraben | 0.10 |
| Sodium metabisulfite | 0.15 |
| Mequinol | 2 |
| Paracetamol | 0.06 |
| Triethanolamine | qs pH 4.5 |

1-10. (canceled)

11. A skin depigmenting composition, comprising:
mequinol,
at least one depigmenting agent, and
a physiologically acceptable vehicle;
wherein the depigmenting agent is selected from the group consisting of kojic acid, derivatives of kojic acid, azelaic acid, derivatives of azelaic acid, dicarboxylic acids, vitamin C, derivatives of vitamin C, arbutin, derivatives of arbutin, paracetamol and mixtures thereof.

12. The skin depigmenting composition as claimed in claim 11;
wherein the mequinol and the depigmenting agent are present in the physiologically acceptable vehicle.

13. The composition as claimed in claim 11, wherein the depigmenting agent is arbutin, paraacetamol or vitamin C.

14. The composition as claimed in claim 11, further comprising:
at least one sunscreen.

15. The composition as claimed in claim 13, further comprising:
at least one sunscreen.

16. The composition as claimed in claim 11, wherein the composition is a medicament.

17. The composition as claimed in claim 13, wherein the composition is a medicament.

18. The composition as claimed in claim 14, wherein the composition is a medicament.

19. The composition as claimed in claim 11, wherein the composition is a cosmetic composition.

20. The composition as claimed in claim 13, wherein the composition is a cosmetic composition.

21. The composition as claimed in claim 14, wherein the composition is a cosmetic composition.

22. A pharmaceutical preparation, comprising:
mequinol,
at least one depigmenting agent, and
a physiologically acceptable vehicle;
wherein the mequinol is present in an amount effective for preventing or treating a pigmentation disorder.

23. The pharmaceutical preparation as claimed in claim 22;
wherein the mequinol and the depigmenting agent are present in the physiologically acceptable vehicle.

24. The pharmaceutical preparation of claim 22;
wherein the pigmentation disorder is melasma.

25. A cosmetic composition, comprising:
mequinol,
at least one depigmenting agent, and
a physiologically acceptable vehicle;
wherein the mequinol is present in an amount effective for protecting against the harmful aspects of sunlight.

26. The cosmetic composition as claimed in claim 25;
wherein the mequinol and the depigmenting agent are present in the physiologically acceptable vehicle.

27. A cosmetic composition comprising:
mequinol,
at least one depigmenting agent, and
a physiologically acceptable vehicle;
wherein the mequinol is present in an amount effective as a skin bleaching agent.

28. The cosmetic composition as claimed in claim 27;
wherein the mequinol and the depigmenting agent are present in the physiologically acceptable vehicle.

29. A method for protection of skin against the harmful aspects of sunlight, comprising:
applying to the skin, a skin depigmenting composition comprising mequinol, at least one depigmenting agent and a physiologically acceptable vehicle.

30. The method for protection of skin against the harmful aspects of sunlight as claimed in claim 29;
wherein the mequinol and the skin depigmenting agent are present in the physiologically acceptable vehicle.

31. A method of bleaching skin, comprising:
applying to the skin, a skin depigmenting composition comprising mequinol, at least one depigmenting agent and a physiologically acceptable vehicle.

32. The method of bleaching skin as claimed in claim 31;
wherein the mequinol and skin depigmenting agent are present in the physiologically acceptable vehicle.

33. A non-therapeutic cosmetic process for either beautifying skin, enhancing the surface appearance of skin, or both beautifying and enhancing the surface appearance of skin comprising:
applying a composition to the skin, to integuments of the skin, or to both the skin and integuments of the skin, wherein the composition comprises mequinol and at least one depigmenting agent.

34. The non-therapeutic cosmetic process as claimed in claim 33;
wherein the mequinol and the depigmenting agent are present in a physiologically acceptable vehicle.
